(19) United States
(12) Patent Application Publication
(10) Pub. No.: US 2019/0208814 A1
(43) Pub. Date: Jul. 11, 2019

(54) ATOMIZER WITH INTERCHANGEABLE ELECTRODES

(71) Applicants: Xingxing Feng, Shenzhen (CN); Cheng Liang Fan, Los Angeles, CA (US)

(72) Inventors: Xingxing Feng, Shenzhen (CN); Cheng Liang Fan, Los Angeles, CA (US)

(73) Assignee: Shenzhen Uwell Technology Limited Company, Shenzhen (CN)

(21) Appl. No.: 15/866,941

(22) Filed: Jan. 10, 2018

(51) Int. Cl. A24F 47/00; A61M 15/06; A61M 11/04; A61M 16/00

(52) U.S. Cl. CPC A24F 47/008; A61M 15/06; A61M 2016/0018; A61M 2205/8206; A61M 11/042

(57) ABSTRACT

An atomizer may comprise a main body, a mouthpiece, and an electrode member. The main body is generally elongated and cylindrical and has an interior space, a first end, and a second end. The mouthpiece coupled with the main body seals the first end, and the interior space is configured to fill with at least one kind of electronic cigarette liquid which is atomized through heat. The electrode member having an electrode is adapted to couple with the second end of the main body to form an enclosed space therebetween. Additionally, the electrode member is interchangeable and each electrode member has a predetermined shape, so an airflow movement of the vapor can be changed according to the shape of the electrode, thus providing a preferable taste of vaping for a user.

### FIELD OF THE INVENTION

The present invention relates to an atomizer and more particularly to an atomizer with interchangeable electrodes.

### BACKGROUND OF THE INVENTION

Since the atomizer, also known as e-cigarette, is introduced to the market, the global usage of atomizer has risen tremendously. The atomizer is invented to help smoker to quit smoking and since an aerosol inhaled by a user is created by heating a liquid through electrical method, it can simultaneously solve secondhand smoke problem. Generally, the atomizer comprises a coil, and an electrode is implanted into the coil. When the atomizer works, the airflow entering the coil is configured to go through the electrode, and the electrode is adapted to change the direction and flowing pattern of the airflow before it is inhaled by a user.

However, the conventional atomizer is disadvantageous because: the electrode of the atomizer cannot be changed, and the atomizer can only provide one kind of vaping taste, which is hard to meet requirements of users having different vital capacities. Therefore, there remains a need for a new and improved design for an atomizer to overcome the problems presented above.

### SUMMARY OF THE INVENTION

The present invention provides an atomizer which comprises a main body, a mouthpiece, and an electrode member. The main body is generally elongated and cylindrical and has an interior space, a first end, and a second end, wherein the first end and the second end are respectively the front end and the rear end of the main body. The mouthpiece is coupled with the main body and seals the first end, and the interior space is configured to fill with at least one kind of electronic cigarette liquid which is atomized through heat. A chimney penetrating through the interior space is connected and communicated with the mouthpiece at one end, and a housing connected and communicated with the chimney at another end thereof is adapted to seal the second end of the main body to only allow gas to pass through. Moreover, the housing is used for axially accommodating a coil member which is configured to generate heat to vaporize the electronic cigarette liquid through the housing. The electrode member having an electrode is adapted to couple with the second end of the main body to form an enclosed space therebetween, and the electrode is inserted into the housing and surrounded by the coil member. Furthermore, an airflow inlet is coupled with a rear end of the housing, and a battery member with a sensor is configured to connect to the electrode member from a rear side thereof to detect air movement generated by a user's inhaling or puffing act, thereby activating the atomization process. Additionally, the electrode member is changeable, and through different shapes of the electrodes of the electrode members, the atomizer can provide different tastes of vaping for a user.

Comparing with conventional atomizer, the present invention is advantageous because: a user can selectively change the electrode member having a preferred shape of the electrode to get different tastes of vaping.

### BRIEF DESCRIPTION OF THE DRAWINGS

FIG. 1 is a sectional assembly view of an atomizer of the present invention.

FIG. 2 is a diagram illustrating different types of electrodes for the atomizer of the present invention.

### DETAILED DESCRIPTION OF THE INVENTION

The detailed description set forth below is intended as a description of the presently exemplary device provided in accordance with aspects of the present invention and is not intended to represent the only forms in which the present invention may be prepared or utilized. It is to be understood, rather, that the same or equivalent functions and components may be accomplished by different embodiments that are also intended to be encompassed within the spirit and scope of the invention.

Unless defined otherwise, all technical and scientific terms used herein have the same meaning as commonly understood to one of ordinary skill in the art to which this invention belongs. Although any methods, devices and materials similar or equivalent to those described can be used in the practice or testing of the invention, the exemplary methods, devices and materials are now described.

All publications mentioned are incorporated by reference for the purpose of describing and disclosing, for example, the designs and methodologies that are described in the publications that might be used in connection with the presently described invention. The publications listed or discussed above, below and throughout the text are provided solely for their disclosure prior to the filing date of the present application. Nothing herein is to be construed as an admission that the inventors are not entitled to antedate such disclosure by virtue of prior invention.

In order to further understand the goal, characteristics and effect of the present invention, a number of embodiments along with the drawings are illustrated as following:

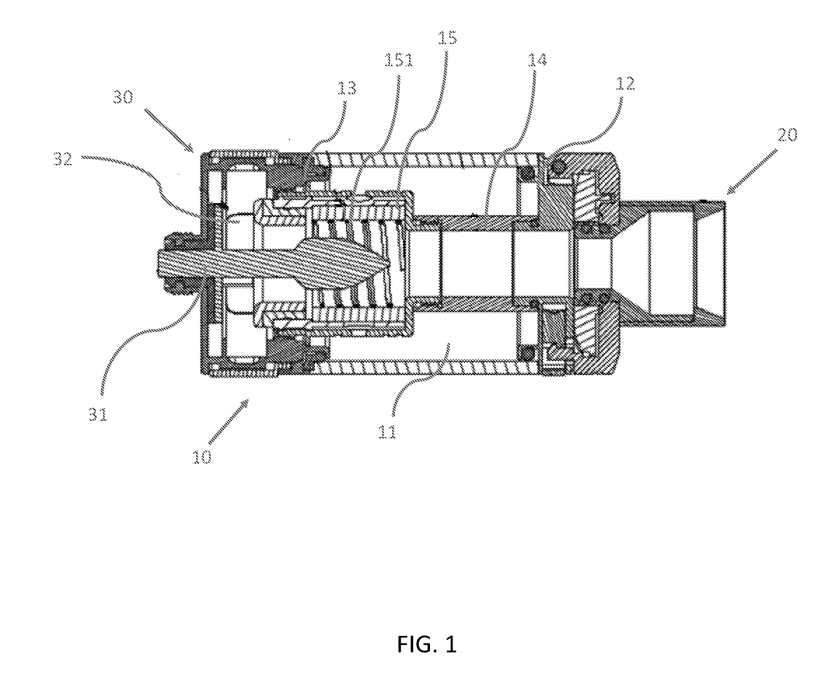

Referring to FIG. 1, the present invention provides an atomizer which comprises a main body (10), a mouthpiece (20), and an electrode member (30). The main body (10) is generally elongated and cylindrical and has an interior space (11), a first end (12), and a second end (13), wherein the first end (12) and the second end (13) are respectively the front end and the rear end of the main body (10). The mouthpiece (20) is coupled with the main body (10) and seals the first end (12), and the interior space (11) is configured to fill with at least one kind of electronic cigarette liquid which is atomized through heat. A chimney (14) penetrating through the interior space (11) is connected and communicated with the mouthpiece (20) at one end, and a housing (15) connected and communicated with the chimney (14) at another end thereof is adapted to seal the second end (13) of the main body (10) to only allow gas to pass through. Moreover, the housing (15) is used for axially accommodating a coil member (151) which is configured to generate heat to vaporize the electronic cigarette liquid through the housing (15). The electrode member (30) having an electrode (31) is adapted to couple with the second end (13) of the main body (10) to form an enclosed space therebetween, and the electrode (31) is inserted into the housing (15) and surrounded by the coil member (151). Furthermore, an airflow inlet (32) is coupled with a rear end of the housing (15), and a battery member with a sensor (not shown in the drawings) is configured to connect to the electrode member (30) from a rear side thereof to detect air movement generated by a user's inhaling or puffing act, thereby activating the atomization process. Additionally, the electrode member (30) is changeable, and through different shapes of the electrodes (31) of the electrode members (30), the atomizer can provide different tastes of vaping for a user.

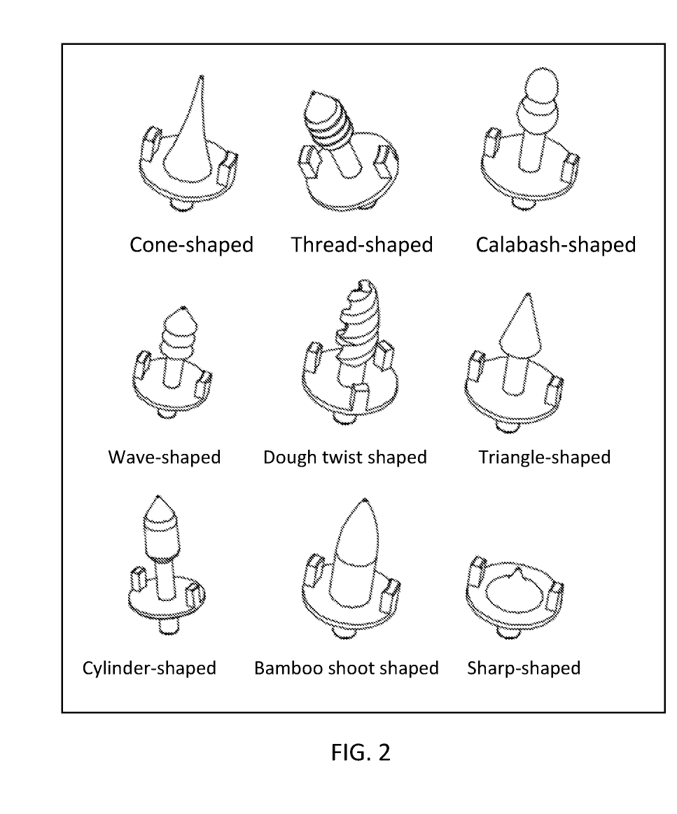

In one embodiment, referring to FIG. 2, the electrode (31) is cone-shaped.

In another embodiment, referring to FIG. 2, the electrode (31) is thread-shaped.

In still another embodiment, referring to FIG. 2, the electrode (31) is calabash-shaped.

In a further embodiment, referring to FIG. 2, the electrode (31) is wave-shaped.

In still a further embodiment, referring to FIG. 2, the electrode (31) is dough twist shaped.

In yet a further embodiment, referring to FIG. 2, the electrode (31) is triangle-shaped.

In a particular embodiment, referring to FIG. 2, the electrode (31) is cylinder-shaped.

In a preferred embodiment, referring to FIG. 2, the electrode (31) is bamboo shoot shaped.

In an advantageous embodiment, referring to FIG. 2, the electrode (31) is sharp-shaped.

In actual application, the e-liquid is filled in the interior space (11) of the main body (10), and when the sensor detects air movement generated by a user's inhaling or puffing act, the activated coil member (151) generates heat to vaporize the e-liquid through the housing (15). Thereafter, the vapor is configured to pass through the second end (13) of the main body (10), the enclosed space between the main body (10) and the electrode member (30) into the airflow inlet (32). Before inhaled by a user through the chimney (14) and the mouthpiece (20), the vapor is adapted to pass through the electrode (31) of the electrode member (30), and the airflow movement of the vapor is configured to be changed according to the shape of the electrode (31). Thus, through the changeable electrode member (30), the atomizer of the present invention can provide different tastes of vaping for the user.

Comparing with conventional atomizer, the present invention is advantageous because: a user can selectively change the electrode member (30) having a preferred shape of the electrode (31) to get different tastes of vaping.

Having described the invention by the description and illustrations above, it should be understood that these are exemplary of the invention and are not to be considered as limiting. Accordingly, the invention is not to be considered as limited by the foregoing description, but includes any equivalents.

What is claimed is:

1. An atomizer comprising a main body, a mouthpiece, and an electrode member, wherein the main body is elongated and cylindrical and has an interior space filled with at least one kind of electronic cigarette liquid, a first end, and a second end; the mouthpiece coupled with the main body seals the first end; and the electrode member has an electrode configured to couple with the second end of the main body to form an enclosed space therebetween, wherein the electrode member is interchangeable and each electrode member has a predetermined shape, so an airflow movement of the vapor is configured to be changed according to the shape of the electrode.
